(19) United States
(12) Patent Application Publication
(10) Pub. No.: US 2017/0146473 A1
(43) Pub. Date: May 25, 2017

(54) MONITORING THE EFFECT OF SUBSTANCES ON IN VITRO TISSUE

(71) Applicant: FRAUNHOFER-GESELLSCHAFT ZUR FOERDERUNG DER ANGEWANDTEN FORSCHUNG E.V., Muenchen (DE)

(72) Inventors: Florian GROEBER, Würzburg (DE); Jan HANSMANN, Eibelstadt (DE); Heike WALLES, Leuttresserbeg (DE)

(73) Assignee: FRAUNHOFER-GESELLSCHAFT ZUR FOERDERUNG DER ANGEWANDTEN FORSCHUNG E.V., Muenchen (DE)

(21) Appl. No.: 15/320,620

(22) PCT Filed: Jun. 18, 2015
(86) PCT No.: PCT/EP2015/063705

§ 371 Date: Dec. 20, 2016

(30) Foreign Application Priority Data
Jun. 25, 2014 (DE) 10 2014 009 537.1

(51) Int. Cl. G01N 27/02; G01N 33/483

(52) U.S. Cl. CPC G01N 27/026; G01N 33/4833

(57) ABSTRACT

The invention relates to methods and means for the non-destructive characterization of biological tissue in vitro and in particular the determination of the effect of substances on said biological tissue or the determination of the maturity or degree of differentiation of said tissue, using non-invasive and in particular recurrently measurable electrical variables. These variables can be calculated, by means of a novel method, from the measured electrical impedance of the biological tissue.

The invention relates to methods and means for the non-destructive characterization of biological tissue in vitro and in particular the determination of the effect of substances on said biological tissue or the determination of the maturity or degree of differentiation of said tissue, using non-invasive and in particular recurrently measurable electrical variables. These variables can be calculated, by means of a novel method, from the measured electrical impedance of the biological tissue.

In basic biological research, and in particular in applied biomedical research, biological tissues cultivated in vitro, in particular multilayer tissues, above all so-called tissue equivalents reconstructed de novo from isolated individual cells, constitute important tools in the investigation of biological, that is to say in particular physiological or cellular, effects of substances and materials. A particular area of application is the calculation of irritative effects of substances on the skin. The provision of and the determination of the effect of substances based on in vitro tissue equivalents is an attractive alternative compared with the experiments on live animals that have been prescribed up to now, both in medical research and in cosmetic production.

In addition to the tissues or biopsies directly explanted from the animal or human body, which can mostly be cultivated for the purpose of medical research in bioreactors, in particular, the tissue equivalents which can be built up de novo in multiple layers from isolated individual cells and/or cell lines can be used in particular for series of tests. At the same time, these in vitro tissue equivalents can also be matched with regard to their composition and/or their cellular structure to the specific test and problem. It is problematic, here, to reproducibly provide these in vitro tissue equivalents (e.g. in vitro skin equivalents) in large numbers and in consistent quality, in particular in structure, maturity or degree of differentiation. It is desirable to have a continuous monitoring and/or final inspection available, which enables reliable conclusions to be drawn relating to the quality of the mass-produced tissue equivalents.

In addition, known non-invasive methods for determining, in particular the quantification of the effect of a substance in, in particular, multilayer, biological in vitro tissues is not sensitive enough to reliably detect and, in particular, also quantify above all irritative or even sub-irritative processes in the in vitro tissues. It is desirable to have available improved, in particular more sensitive and with higher temporal resolution, non-invasive monitoring methods for identifying and/or quantifying the effect of substances and the kinetics of their effect, which allow more accurate and more extensive conclusions to be drawn.

On the one hand, the invention is based on the problem of providing an improved, non-destructive method for determining processes in in vitro cultivated biological tissues. Associated with this, the technical problem is the provision of methods and means for characterizing an in vitro tissue based on variables. In this regard, there is also the problem of providing methods and means which can demonstrate particularly sensitively the biological effect of substances on in vitro cultivated multilayer biological tissue. Associated with this is also the technical problem of providing methods and means which enable a non-invasive, that is to say in particular ongoing, monitoring or a non-destructive final process inspection of the state of standardized biological tissues to be used for test purposes in vitro, in particular in vitro tissue equivalents. At the same time, in particular, it must be possible to easily and reliably determine the degree of cellular differentiation or degree of maturity of the biological tissue. In addition, a high temporal resolution, that is to say high sampling rates, must be achieved during the measurement.

The present invention completely solves the basic technical problem by the provision of a method for characterizing a biological tissue according to claim 1 and of means which are specifically designed to be able to carry out the method according to the invention.

The subject matter of the invention is, in particular, a method for characterizing an, in particular multilayer, biological tissue in vitro containing the following steps:

a) Measurement of the electrical impedance of the biological tissue as a function of frequency, wherein an impedance spectrum Z(ω) is obtained, and
b) Iterative matching of n model spectra Zₘ(ω)n to the measured impedance spectrum Z(ω), wherein each model spectrum Zₘ(ω)n is in each case determined by a formula which is selected from the family of formulae with n formulae according to Formula (1):

Zₘ(ω)n=R_{S}+Z_{CPE}(ω)+ΣZ_{Cell}(ω)i, for i=1 to n (1)

where for each i
Z_{Cell}(ω)i represents the specific impedance of an electrically active layer i in the tissue, wherein every electrically active layer i in each case corresponds to a network in the form of a parallel circuit of the ohmic component R_{Cell}i of this layer and the real capacitive component C_{Cell}(ω)(Ni)i of this layer according to Formula (2):

ZCell(ω)i=R_{Cell}i∥C_{Cell}(ω)(Ni)i (2)

and where

Ni is the ideality of this real capacitive component C_{Cell}(ω)(Ni)i, and

c) Determination for each of the n model spectra Zₘ(ω)n the model spectrum which for each n is optimally matched to the measured impedance spectrum Z(ω),
d) Calculation of at least one variable selected from ohmic component R_{Cell} and real capacitive component C_{Cell}(ω)(N) of each of the matched n model spectra as the variable which characterizes the biological tissue.

Preferably, in doing so, it is provided in Step b), for each n, to approximate the model spectra Zₘ(ω)n in each case to the impedance spectrum Z(ω) measured in Step a) by automatic variation of at least one of the impedance-determining components R_{Cell}i and C_{Cell}(ω)(Ni)i of each electrically active layer i.

Preferably, in doing so, it is provided that the iterative matching in Step b) is carried out automatically using the method of least mean squares. In Step c), for each n, the optimally matched model in each case is characterized in that it has the smallest residues compared with the measured impedance spectrum in each case.

In this way, an optimally matched model spectrum which optimally represents the measured impedance spectrum is found for each of the n models. In a variant, it is provided that the number of electrically active layers, which is expedient for the, in particular multilayer, tissue to be investigated, be specified in each case at the beginning or at the end of the automatic analysis to determine the model spectra and the variables obtainable therefrom by choosing the number n. If, for example, as a result of biological cell knowledge, histological findings and/or earlier measurements in a multilayer tissue, two electrically active layers are expected, the number n=2 can be chosen in order to limit the analysis to a model of exactly two layers i. If, for example, in a multilayer tissue, a single prominent layer with prominent electrical properties with regard to the impedance of the whole tissue is indicated, the number n=1 can be chosen to limit the analysis to a model with exactly one layer i. Preferably, therefore, a so to speak cursory method, wherein n=1 or n=2, is chosen. With this cursory approach of the analysis according to the invention, in many cases a sufficiently accurate and sensitive characterization of the effect of a substance on biological tissues can be made with the variables so found, wherein, at the same time, the resource outlay for electronic data processing can be kept small.

In an alternative preferred variant, it is provided that, in Step c), from the n optimally matched models Zₘ(ω)n found, the one which when viewed over all n models has the smallest deviations from the impedance spectrum Z(ω) measured is chosen as the best matched model Zₘ(ω), and in Step d), the at least one variable is calculated from this best matched model spectrum. As a result of this automated matching of the models, including with regard to the number of the electrically active layers i contained in the model, those variables which best characterize the biological tissue can be automatically calculated and, at the same time, a conclusion can be drawn regarding the actually existing number of electrically active layers in the biological tissue.

Accordingly, the invention preferably provides that, in Step d), for each electrically active layer i of the best matched model spectrum found, at least one variable, selected from ohmic component R_{Cell}i and capacitive component C_{Cell}(ω)(Ni)i, is calculated separately as a variable which accurately characterizes this electrically active layer i of the biological tissue.

Accordingly, it is preferably also provided that, in the formula Zₘ(ω)n of the best matched model spectrum found, n indicates the number of electrically active layers actually prevailing in the biological tissue.

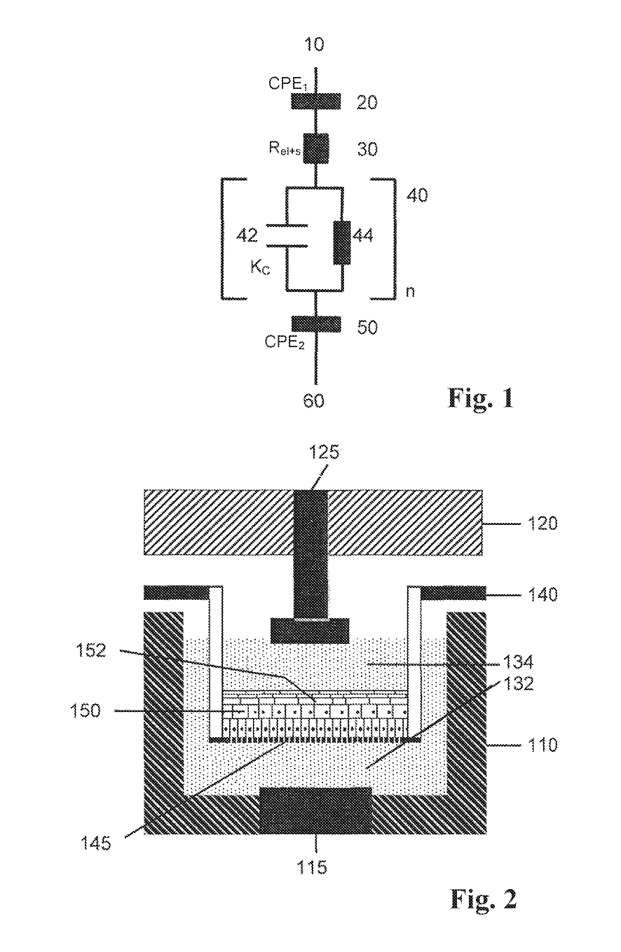

The method according to the invention therefore especially provides for certain electrical variables, which allow robust conclusions to be drawn relating to the physiological state of the biological tissue, to be extracted from an electrical impedance spectrum, which is calculated non-invasively from the biological tissue in a way which is known per se, by modeling. A novel model of the circuit of the main electrical components in a biological tissue is used for the purpose of modeling. It is understood that the tissue is substantially cultivated or fixed on a membrane or similar and divides a bioreactor chamber into an upper (apical) and a lower (basal) chamber. The electrical impedance is measured along the apical to basal vector, that is to say perpendicular to the plane of the biological tissue. The impedance measurement electrodes are located, on the one hand, in the apical chamber of the bioreactor and, on the other, in the basal chamber of the bioreactor in a manner which is known per se. Correspondingly, the equivalent circuit diagram of the biological tissue extends along these two reference points. The equivalent circuit diagram of a tissue on which the invention is based is shown in FIG. 1. It consists essentially of a combined and substantially frequency-independent series resistor Rₛ connected in series with a combined so-called constant phase element, which is frequency-dependent but is substantially independent of the biological processes in the biological tissues and therefore does not contribute to the variables calculated according to the invention. Also connected in series is at least one network consisting of a parallel circuit of an ohmic component of an electrically relevant layer in the biological tissue and a capacitive component of this layer. In contrast to the teaching of the prior art, the model uses a so-called real capacitor for the capacitive component. A real capacitor is distinguished by an ideality N<1 which, without wishing to be tied to a theory, takes into account the “roughness” of the capacitor. Accordingly, the impedance portion of this network of an electrically active layer can be calculated in accordance with Formula (2):

ZCell(ω)i=R_{Cell}∥C_{Cell}(ω)(Ni)i (2)

According to the invention, the equivalent circuit diagram now additionally provides the possibility that at least one of these networks, or even a plurality of networks (n>1), can be connected in series. According to the invention, an electrically active layer in the biological tissues is characterized by such a network. Accordingly, the total impedance of the electrical equivalent circuit diagram, on which the present invention is based, is calculated depending on the number n of existing electrically active layers in the multilayer biological tissue according to:

Zₘ(ω)n=R_{S}+Z_{CPE}(ω)+Z_{Cell}(ω)i, for i=1 to n (1)

In order to analyze the measured impedance spectrum, the modeled characteristic of the electrical impedance resulting from the chosen electrical equivalent circuit diagram (model spectrum) is now matched, that is to say fitted, to the measured impedance spectrum in a manner which is known per se. At least the capacitive component and/or the ohmic component of an electrically active layer of a corresponding parallel network in the electrical equivalent circuit diagram are calculated as the characteristic variables according to the invention. At the same time, it is preferably provided that, for each electrically active layer (n>2), the effective “layer” corresponds to a certain cell layer. In other cases, the electrically active layer is formed by other, possibly non-cellular, structures in the biological tissue. Examples of these are corneous layers, connective tissue layers, vessel layers or prominently distinctive basal membranes. A distinctive extra-cellular matrix present in the biological tissue can also form such an electrically active layer. The invention therefore provides for the use of an equivalent circuit diagram for analyzing impedance spectra which make use of the idea of the so-called electrically active layer. A specific example is an explanted in vitro cultivated cornea of a vertebrate's eye, for example, which consists substantially of an epithelial layer with an endothelial layer beneath it. The epithelial layer and the endothelial layer in each case form an electrically active layer, which can be differentiated in the equivalent circuit diagram according to the invention.

The subject matter of the invention is therefore a method for characterizing a biological tissue in vitro containing the following steps: a) Measurement of the electrical impedance of the biological tissue as a function of frequency, wherein an impedance spectrum Z(ω) is obtained, b) matching to the measured impedance spectrum Z(ω), wherein a model spectrum Zm(ω)n is determined by a formula which is selected from the family of formulae: Zₘ(ω)n=R_{S}+Z_{CPE}(ω)+Sum of (Z_{Cell}(ω)i) for i=1 to n, where n Z_{Cell}(ω)i is the impedance of an electrically active layer i in the tissue which corresponds to a parallel connection of the ohmic component R_{Cell}i of this layer and the real capacitive component C_{Cell}(ω)(Ni)i of this layer: Z_{Cell}(ω)i=R_{Cell}i in parallel with C_{Cell}(ω)(Ni)i and where Ni is the ideality of this real capacitive component C_{Cell}(ω)(Ni)i and is always less than 1, c) determining, for each model spectrum Zₘ(ω)n, the model spectrum which in each case is optimally matched to the measured impedance spectrum Z(ω), d) calculating at least one variable selected from ohmic component R_{Cell} and capacitive component of each of the matched model spectra as the variable which characterizes the biological tissue, wherein, preferably, in Step b), for each n, the model spectra Zₘ(ω)n are in each case approximated to the impedance spectrum Z(ω) measured in Step a) by variation of at least one of the impedance-determining components R_{Cell}i and capacitive component C_{Cell}(ω)(Ni)i of each electrically active layer i in each case, and/or wherein, preferably, the interactive matching in Step b) is carried out using the method of least mean squares and, in Step c), for each n, the optimally matched model in each case is characterized in that it has the smallest residues compared with the measured impedance spectrum in each case.

In this regard, the invention particularly provides for selectively analyzing the electrical variables of a specific electrically active layer separately, in the case of the cornea the epithelial layer or the endothelial layer. Another specific example is the epithelial layer of the skin or a skin equivalent which consists of a corneous layer (stratum corneum) and epithelial cells which lie beneath it. A first electrically active layer is formed by this non-vital corneous layer, and a second electrically active layer by the epithelial cell layer. In a young skin tissue or skin equivalent, this corneous layer is still comparatively thin and, with regard to the magnitude of the electrical variables, constitutes a comparable electrically active layer compared with the epithelial cell layer. On the other hand, the mature skin or a mature skin equivalent has a thicker corneous layer which comes to the fore compared with the epithelial layer with regard to the magnitude of the electrical variables. In a model according to the invention, young, immature skin epithelium would be best represented by an equivalent circuit diagram which has two, in particular equally weighted, electrically active layers (n=2), whereas mature skin epithelium with thick corneous layer is represented by an equivalent circuit diagram with a single prominent electrically active layer (n=1).

In conjunction with the present invention, a “multilayer” biological tissue is not necessarily to be understood as a tissue that has two, anatomically differentiatable and above all different vital cell layers, for example keratinocytes and fibroblasts. It is also understood to mean tissues in which layers of vital cells and not vital layers are present, for example a corneous layer (stratum corneum). Furthermore, it is understood that an anatomically or histologically definable layer of a biological tissue does not necessarily correspond to a single so-called electrically active layer according to the equivalent circuit diagram according to the invention. A plurality of histologically differentiatable layers of a multilayer tissue can therefore also be represented by a single electrically active layer.

In a particular embodiment, the invention allows the acute influence of active substances and also changes within the framework of maturity and differentiation of the tissue on certain layers to be limited or to be selectively investigated there.

Preferably, the biological tissue to be investigated is selected from skin equivalent reconstituted de novo from isolated cells of the human or animal body and/or from cell lines and explanted ex vivo tissue (biopsy) of the human or animal body; in a particular embodiment of the invention, the biological tissue is a so-called in vitro skin equivalent as has been described in particular in DE 10 062 623 A1 and DE 10 2010 023 156 A1, the disclosed content in this regard of which is hereby to be included in the disclosure of the invention.

However, the invention is not restricted to the application with skin tissues. Other preferred ex vivo tissues and in vitro tissue equivalents that can be used are chosen from intestinal epithelium, the retina, the cornea, kidney tissue, liver tissue and others.

A specific application of this invention is the characterization, classification and, in particular, quantification of sub-irritative, irritative and/or corrosive effects of substances on the biological tissue. A specific application of this is the determination of corrosive and/or irritative effects of test substances on the skin which, in the form of in vitro skin equivalents, can be carried out reliably and sensitively by means of the method according to the invention and/or the means according to the invention. Above all, the invention allows a more accurate quantification and classification of the effects of substances than was previously possible with known measuring and analytical methods. Advantageously, a corrosive effect, that is to say an irreversible damage to the tissue, can be differentiated from an irritative effect, that is to say a reversible impairment to the tissue, and from a sub-irritative effect, that is to say a physiological effect of the tissue without damage. Such classifications are expedient, particularly in conjunction with the investigation of the effect of substances on the skin in form of in vitro skin equivalents, wherein, in particular, great importance is attached to the reliable detection of the sub-irritative effect of a substance, which is generally described as “burn,” “itch” or “sting.”

For example, in conjunction with the invention, it has been shown that a sub-irritative effect which is associated with the sensations described above is predominantly caused by processes in non-vital layers, here the corneous layer of the epithelial cell layer. For example, sub-irritative acting solvents such as 2-propanol act predominantly in the non-vital corneous layer in that they increase the solubility of barrier-forming components here, as a result of which the corneous layer becomes more permeable. This can be detected, and advantageously also quantified, by means of the method according to the invention. This means the changes observed in electrical variables in the tissue are not necessarily due to biological processes within layers of vital cells but can also be caused by purely chemical-physical processes in non-vital layers.

In conjunction with the investigation of skin tissue, in particular in the form of in vitro skin equivalents, surprisingly it has been shown that a highly irritative or corrosive action of a substance is associated with a significant reduction in the “ohmic resistance” variable in the electrically relevant layer and with a significant increase in the “real capacitance” variable in this layer. In contrast with this, although a weakly irritative action likewise effects a significant reduction in the ohmic resistance, the real capacitance is changed to a far lesser extent and the variables return substantially to their original values in the course of time after the end of the substance action, which characterizes reversibility of the damage. In the case of sub-irritative action of a substance, an observable change occurs only or substantially exclusively in the ohmic resistance. In addition, these are only very shortly after the initial contact and are subsequently fully reversible. Because of the robustness of the analysis results, the method according to the invention enables statistically resilient conclusions relating to the classification of the effect of the substance and also to dose/effect relationships to be drawn even with low sample numbers.

The invention is based on the analysis of data of an electrical impedance measurement. The impedance measurement on the tissue can be carried out in a manner which is known per se. Preferably, in Step a), the impedance spectrum of the biological tissue is measured by imposing an alternating current with alternating frequency components, preferably in the range from 1 Hz to 100 kHz, more preferably from 20 Hz to 20 kHz, and measuring the frequency-dependent alternating voltage which drops across the tissue layers. Preferably, an alternating current of approximately 2 to 5 mA, preferably a maximum of approximately 3 mA, is imposed and the resulting frequency-dependent voltage drop measured.

In an alternative variant of the impedance measurement, in Step a), the impedance spectrum of the biological tissue is measured by applying an alternating voltage with alternating frequency components, preferably in the range from 1 Hz to 100 kHz, more preferably from 20 Hz to 20 kHz, across the tissue layers and measuring the frequency-dependent alternating current which flows as a result.

As is known, alternating voltages or alternating currents with substantially sinusoidal characteristic and discrete individual frequencies are used to calculate the electrical impedance spectrum. In doing so, impedance spectra are then calculated by individual measurement at different discrete frequencies which, for example, are tuned in octave steps or decade steps. However, the invention also provides for the use of alternating voltages or currents with characteristics containing harmonics, with structured noise and/or transient pulses, as a result of which the impedance can be measured simultaneously in a plurality of frequency components and registered by appropriate analysis methods such as FFT and others. The parallel, simultaneous calculation of the impedance at a plurality of frequencies enables the measuring time to be shortened and/or disadvantageous activity at the electrode materials or surfaces, which can affect the measuring result, to be avoided or reduced.

In an alternative embodiment, the electrical impedance of the biological tissue is measured at only a few selected frequencies in the range from 1 Hz to 100 kHz, for example 100 Hz and 1000 Hz, in particular at a single frequency, for example 100 Hz. In this embodiment, the analysis method according to the invention manages without the otherwise necessary curve fit of the modeled spectrum to the measured model spectrum. Rather, here, the biological variables are extracted directly from the measured data by specifying a certain selected equivalent circuit diagram, for example by specifying with regard to the electrically active layers and, alternatively or additionally, by specifying the magnitude of electrical series resistance and constant phase element (CPE) within the measuring arrangement.

Subject matter of the invention is also a method for characterizing the biological effect of an active substance on a multilayer biological tissue in vitro containing the following steps:

A) Initial calculation of at least one first biological variable R_{Cell} and/or C_{Cell}(ω)(N) according to the method according to one of the preceding claims for the biological tissue or a group of biological tissues,
B) Bringing this biological tissue or this group of biological tissues into contact with the active substance, wherein tissue treated with the active substance or a treated group of such biological tissues is obtained, and
C) Re-calculation of the at least one biological variable R_{Cell} and/or C_{Cell}(ω)(N) according to the method according to one of the preceding claims for the treated biological tissue or the treated group of biological tissues,
D) Comparison of the at least one first calculated biological variable before bringing into contact with the at least one re-calculated second biological variable after bringing into contact, wherein a change in the at least one biological variable between first and repeated calculation indicates a biological effect of this active substance on the biological tissue.

Subject matter of the invention is also a device for determining biological variables of a multilayer biological tissue in vitro which is specially designed to carry out the measuring and analysis method according to the invention. For this purpose, the device includes at least the following components:

1) Measuring unit for measuring an impedance spectrum Z(ω) according to Step a) of the method according to one of claims 1 to 10, and
2) Arithmetic unit which is specifically programmed to automatically carry out the analysis method according to the invention as characterized particularly in Steps b) to d) of the method disclosed herein. For this purpose, the arithmetic unit can execute at least the following program steps:
Iterative matching of n model spectra Zₘ(ω)n obtained by modeling electrical variables to the measured impedance spectrum according to Step b) of the method,
Determination of matched model spectra according to Step c) of the method, and
Calculation of at least one variable of the biological tissue according to Step d) of the method.

Preferably, the device additionally includes at least one bioreactor for cultivating biological tissue in vitro with electrodes for applying voltage/current and measuring the impedance over the cultivated biological tissue.

Preferably, the bioreactor includes a plurality of compartments for parallel separate cultivation of a plurality of biological tissues in vitro, and electrodes which are associated with each compartment individually.

Preferably, at the same time, the measuring unit additionally has a multiplexer which connects a plurality of electrodes of the bioreactor to the measuring unit for sequential measurement of the impedance in the plurality of parallel cultivated biological tissues.

A bioreactor provides sterile conditions in which the biological tissues can be measured. Preferred is a bioreactor with eight identical measuring chambers for determining the impedance spectra of eight biological tissues cultivated therein. The bioreactor preferably consists of a bioreactor base plate and a bioreactor cover made, in a manner which is known per se, from plastic material (in particular PEEK). Such a bioreactor is known, for example, from DE 10 2009 022 345 A1, the disclosed content in this regard of which is included in the disclosure of the invention. In the particular embodiment, the bioreactor provides that each biological tissue is cultivated on a culture membrane which is clamped in a separate frame or carrier, a so-called insert. This insert is in each case placed in one of the plurality of separate chambers present in the bioreactor. In a particular embodiment, means and measures are provided in the bioreactor which enable the biological tissue to be positioned in the insert and the insert to be positioned centrally over the electrodes present in the bioreactor. Preferably, it is provided that the biological tissue is positioned centrally in the electrical field of each electrode.

The electrodes of the bioreactor are preferably metal electrodes. In a variant, the electrodes are coated electrodes, which are known per se, with reduced contact potential, for example Ag/AgCl electrodes. In an alternative and preferred variant, the electrodes are made of corrosion-resistant stainless steel material. In particular here, it is provided that the electrode surface be smoothly polished, for example by electro polishing. At the same time, surprisingly it has been established that the measurement of the electrical impedance is improved by reducing the roughness of the surface of the electro-polished electrodes.

In the bioreactor, which is preferably formed in two parts with base part and cover part, it is preferably provided that the working electrodes are formed in the top reactor half (cover part). In doing so, it is preferred that the top working electrodes, which preferably have a cylindrical shape, are immersed in a cup-shaped cell culture insert in each compartment of the bioreactor so that the electrode surface in each case comes to lie as close as possible to the surface of the biological tissue. Preferably, the working electrode is formed in the shape of a die which immerses in the reactor chamber. While each compartment of the bioreactor has a separate working electrode, the counter electrode can likewise either be formed separately for each compartment or formed in the base of the bioreactor as a continuous common counter electrode which extends over a plurality of compartments. Preferably, it is provided that the counter electrode be permanently integrated in the base of the bioreactor. This is designed as a plate electrode, for example, and preferably encompasses the base of a plurality of compartments. A preferred embodiment of the design of the bioreactor and of a compartment thereof is shown in FIG. 2.

Subject matter of the invention is also the use of the device described herein for the purpose of determining the biological effect of test substances on in vitro cultivated multilayer biological tissue based on the change in the calculated electrical variables.

Subject matter of the invention is also the use of the device described herein for the purpose of determining the maturity and degree of differentiation of in vitro cultivated multilayer biological tissue based on the calculated electrical variables.

Finally, subject matter of the invention is also a computer program product including the instructions for automatically carrying out the method characterized in the Steps b) to d) described herein.

The invention is described in more detail by means of the following figures and exemplary embodiments without these being understood to be restricting.

FIG. 1 shows the technical equivalent circuit diagram of a biological tissue, on which the data analysis of the present invention is based, in conjunction with the electrical impedance measurement. From the apical pole 10 (working electrode) of the biological tissue to its basal pole 60 (counter electrode), the equivalent circuit diagram thereof contains an apical constant phase element 20, an ohmic series resistance 30, and, for each electrically active layer in the biological tissue, a network 40 comprising capacitive component 42 and ohmic component 44 which are connected in parallel and, in series downstream thereof, the basal constant phase element 50.

According to the model, the electrical series resistance 30 represents collectively all frequency-independent ohmic series resistances of the measuring setup, including line resistances in the feed wires and electrodes; the resistance of the electrolyte solution (culture medium) is likewise a component of the electrical series resistance 30. Apical and basal constant phase element 20, 50 in each case represent frequency-dependent phenomena in the system, in particular processes at the interfaces of the active electrode and counter electrode, respectively. According to this model, these are independent of biological effects and substance effects; they can be summarized as common constant phase element (CPE). Biologically relevant variables, which, according to the invention, characterize the biological tissue and indicate biological processes, are the ohmic component 44 and the capacitive component 42 of the network 40 of each electrically active layer i of the biological tissue. If, for example, the biological tissue has a single electrically active biological layer (n=1), then the equivalent circuit diagram has a single network 40. If a multilayer biological tissue has two or more electrically active layers, then the equivalent circuit diagram for each electrically active layer i has in each case a separate network 40 which is connected in series with the other components.

The associated ohmic and capacitive components 42, 44 can each have different values for each network 40. In a simplified alternative model according to the invention, the electrical parameters of ohmic and capacitive components 42, 44 are set equal for each network 40.

For a further simplified network according to the invention, the number n=2 and therefore the equivalent circuit diagram has exactly two networks 40 connected in series. In an alternative simplified variant of the invention, the electrical equivalent circuit diagram has exactly one network 40 (n=1).

Characteristic for the equivalent circuit diagram according to the invention is that the capacitive component 42, which in each case is present in the networks 40, is modeled as a real capacitance with real surface properties. Here, the ideality N is <1.

FIG. 2 shows a schematic design of a bioreactor or of a compartment thereof with base part 110 and cover part 120 which forms at least one cell culture chamber 130, in which a cell culture insert 140, which carries the cell culture membrane 145, is suspended. For illustration, a multilayer tissue 150 is shown placed on the cell culture membrane 145 by way of example. An apical electrically active layer 152 is formed in the tissue 150 (shown here by way of example).

The basal culture medium 132 is electrically isolated from the apical culture medium 134. An electrical counter electrode 115 is fitted in the base part 110. A die-shaped working electrode 125, which, in the assembled state of the bioreactor, projects into the lumen of the chamber 130 and, in particular, directly into the cell culture insert 140, is present in the cover part 120. The impedance is measured between counter electrode 115 and working electrode 125 in the biological tissue 150 placed between the electrodes.

In a bioreactor, at least one reactor chamber 130, in which in each case the biological tissue 150 can be cultivated, is formed in the base part 110.

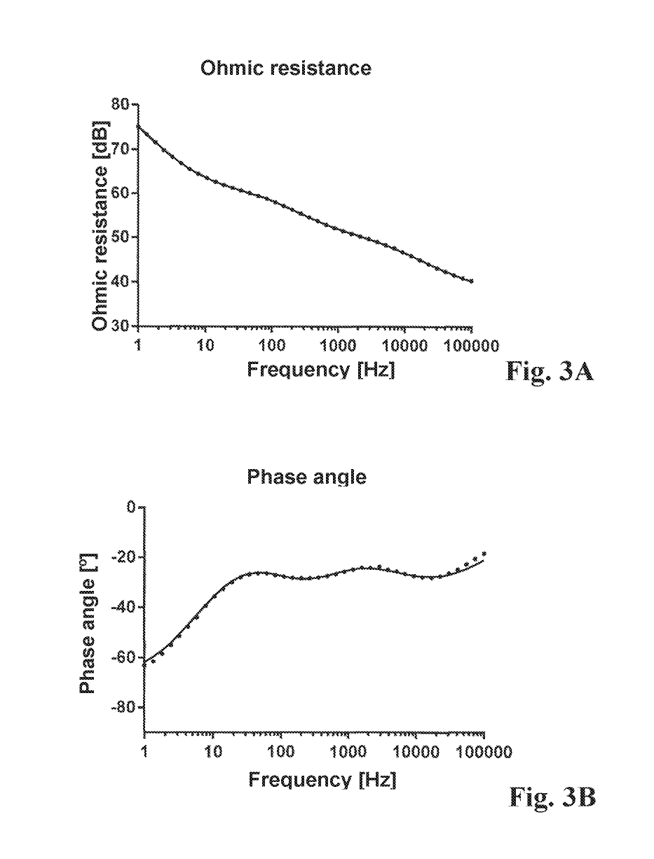

FIG. 3 shows by way of example an electrical impedance spectrum measured on a multilayer biological tissue plotted as Bode diagrams of amplitude (magnitude of the complex resistance) and phase angle. The measured impedance spectrum is in each case represented by the specific measuring points. The best matched model spectrum modeled in accordance with the equivalent circuit diagram according to the invention is superimposed. The fit is made using the “least mean square” method.

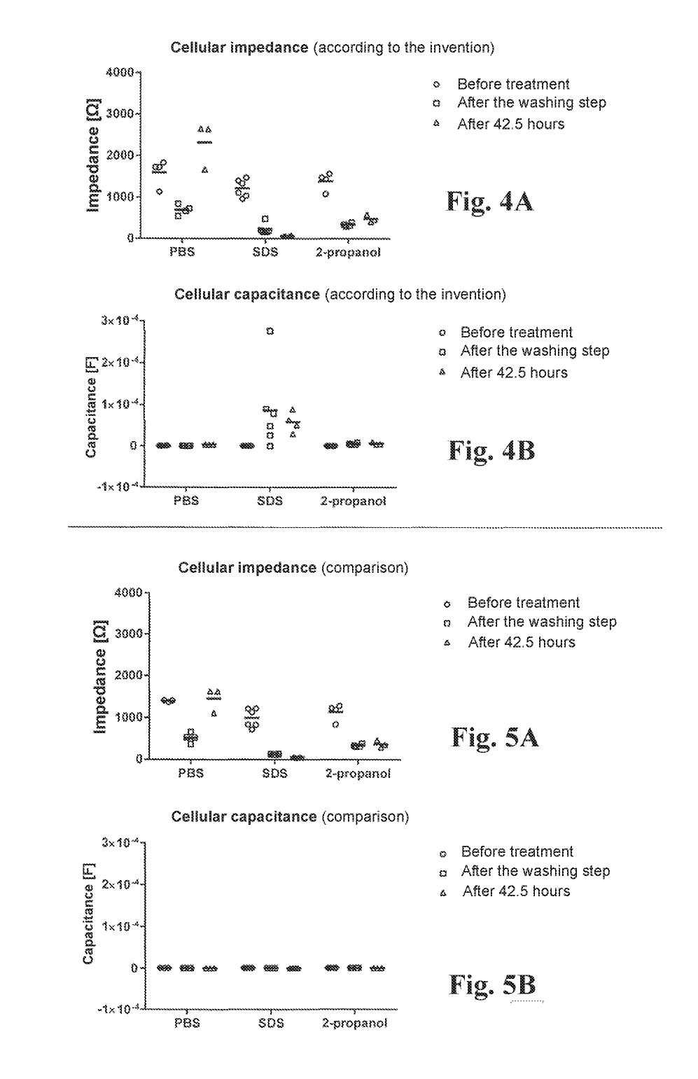

FIG. 4 shows the variables calculated by means of the method according to the invention of biological impedance and biological capacitance of the epithelial layer with stratum corneum of a skin equivalent before treatment with non-irritative substance (PBS), highly irritative substance (SDS) or sub-irritative substance (2-propanol) for approximately 35 min, after an initial washing step and after recovery after approximately 42.5 h. FIG. 4A shows the cellular impedance, FIG. 4B shows the capacitance.

FIG. 5 shows variables calculated after a comparison process (n=1, N=1) from the same measured impedance spectra as in FIG. 4. FIG. 5A shows the cellular impedance, FIG. 5B shows the cellular capacitance.

### EXAMPLE: VERIFICATION OF SUB-IRRITATIVE EFFECTS OF SUBSTANCES IN BIOLOGICAL TISSUES

Multilayer in vitro skin equivalents are reconstituted in a manner which is known per se from isolated cells, namely primary fibroblasts and keratinocytes. In detail, in a first step, the dermal part of the skin equivalent is built up, wherein primary fibroblasts are integrated in a collagen matrix with predominantly collagen type I by suspension. In a second step, the so-called dermis is overlaid with keratinocytes, which then form the epidermal layer. As an option, the formed collagen matrix can be overlaid with fibronectin before applying the keratinocytes in order to form a membrane. The construction of the in vitro skin equivalent takes a total of approximately 21 days. In the last cultivation phase, the plurality of skin equivalents are in each case located in cell culture inserts, which are known per se, in a bioreactor, in the base part of which a planar counter electrode is fitted. In the top cover part is located a die-shaped working electrode which is immersed in the apical culture medium. Apical cell culture medium and basal cell culture medium are in each case electrically isolated from one another and only come into contact with one another via the in vitro skin equivalent.

In order to measure the electrical impedance of the individual skin equivalents in the bioreactor, an alternating current with alternating frequency with an amplitude of approximately 3 mA is applied between apical working electrode, which constitutes the electrical plus pole, and basal counter electrode, which constitutes the electrical minus pole. The frequency is varied in 40 logarithmically scaled steps from 1 Hz to 100 kHz. Sinusoidal alternating currents with discrete frequency are used.

FIG. 4 shows the variables which are calculated in this way for treatment with three known model substances for irritative effects (in accordance with CLP regulation of the European Union or the GHS standard):
1. Phosphate-buffered saline (PBS), which represents an example of non-irritative substances,
2. 2-propanol, which represents an example of sub-irritative substances,
3. Sodium dodecyl sulfate 5% (SDS), which represents an example of highly irritative and abrasive substances.

To investigate the effect of the substance on the skin equivalent, the electrical impedance is in each case measured before treatment (control measurement) and immediately after bringing the skin equivalent into contact with one of the control substances for approximately 35 min and subsequent multiple washing with culture medium (first measurement). A further measurement is carried out after a so-called recovery phase, approximately 42.5 hours after the initial contact with the control substance (second measurement).

For each measured impedance spectrum, the electrical variables according to the invention are calculated by fitting the modeled spectra according to the invention to the measured impedance spectra in each case.

In a control approach, in order to calculate electrical variables, the same measured impedance spectra (control, first measurement, second measurement) are fitted to models which in all cases provide only one single electrically active layer (n=1) and wherein the capacitive component representing the biological tissue is represented by an ideal capacitor without taking the ideality N into account (N=1). FIGS. 5A and 5B show the comparative data calculated in this way.

From the comparison of the analysis method according to the invention, a comparison method shows that in particular a sub-irritative effect, here represented by contact with the substance 2-propanol, cannot be detected using the comparison method. In addition, the analysis method according to the invention also enables a quantification of the irritative effect of substances, whereas, due to its low sensitivity, the comparison method does not enable irritative and sub-irritative effects of substances to be quantified sufficiently meaningfully, as no significant dose-effect relationship can be established here.

1-18. (canceled)

19. A method for characterizing a biological tissue in vitro, the method comprising:
a) measuring an electrical impedance of the biological tissue as a function of frequency, wherein a measured impedance spectrum Z(ω) is obtained;
b) matching of n model spectra Zₘ(ω)n to the measured impedance spectrum Z(ω), wherein each Zₘ(ω)n is determined by a formula selected from the family of formulae:
Zₘ(ω)n=R_{S}+Z_{CPE}(ω)+Sum of (Z_{Cell}(ω)i) for i=1 to n,
where for each i
Z_{Cell}(ω)i is the impedance of an electrically active layer i in the tissue which corresponds to a parallel connection of an ohmic component R_{Cell}i of this layer and a real capacitive component C_{Cell}(ω)(Ni)i of this layer according to the formula:
Z_{Cell}(ω)i=R_{Cell}i∥C_{Cell}(ω)(Ni)i
and where
Ni is the ideality of the real capacitive component C_{Cell}(ω)(Ni)i and is always less than 1;
wherein, for each n, the model spectra Zm(ω)n are in each case approximated to the impedance spectrum Z(ω) measured in Step a) by variation of at least one of the impedance-determining components R_{Cell}i and capacitive component C_{Cell}(ω)(Ni)i of each electrically active layer i in each case;
c) determining for each model spectrum Zₘ(ω)n the model spectrum which for each n is optimally matched to the measured impedance spectrum Z(ω), wherein the optimally matched model in each case has a smallest residue compared with the measured impedance spectrum in each case; and
d) calculating at least one variable selected from ohmic component R_{Cell} and real capacitive component of each optimally matched model spectrum as the variable which characterizes the biological tissue.

20. The method according to claim 19, wherein iterative matching in Step b) is carried out using a least mean squares method.

21. The method according to claim 19, wherein in Step c), from the n optimally matched models Zₘ(ω)n found, the one which when viewed over all n models has the smallest deviations from the impedance spectrum Z(ω) measured is chosen as the best matched model Z(ω), and in Step d), the at least one variable is calculated from this best matched model spectrum Z(ω).

22. The method according to claim 21, wherein in Step d), for each electrically active layer i of the best matched model spectrum found, at least one variable, selected from ohmic component R_{Cell}i and capacitive component C_{Cell}(ω)(Ni)i, is calculated separately as a variable which accurately characterizes this electrically active layer i of the biological tissue.

23. The method according to claim 21, wherein in the formula Zₘ(ω)n of the best matched model spectrum found, n indicates the number of electrically active layers actually prevailing in the biological tissue.

24. The method according to claim 19, wherein n=1 or n=2.

25. The method according to claim 19, wherein in Step a), the impedance spectrum of the biological tissue is measured by imposing an alternating current with alternating frequency components in the range from 1 Hz to 100 kHz and measuring the frequency-dependent alternating voltage which drops across the tissue layers.

26. The method according to claim 19, wherein in Step a), the impedance spectrum of the biological tissue is measured by applying an alternating voltage with alternating frequency components in the range from 1 Hz to 100 kHz across the tissue layers and measuring the frequency-dependent alternating current flowing as a result.

27. The method according to claim 19, wherein the multilayer biological tissue is selected from:
tissue equivalent reconstituted de novo from isolated cells of a human or an animal body and/or from cell lines, and
explanted ex vivo tissue of the human or animal body.

28. A method for characterizing an effect of an active substance on a biological tissue in vitro, the method comprising:
a) initial calculating of at least one first biological variable R_{Cell} and/or C_{Cell}(ω)(N) according to the method of claim 19 for the biological tissue or a group of biological tissues,
b) bringing the biological tissue or group of biological tissues into contact with the active substance, wherein tissue treated with the active substance or a treated group of such biological tissues is obtained,
c) recalculating the at least one biological variable R_{Cell} and/or C_{Cell}(ω)(N) according to the method of claim 19 for the treated biological tissue or the treated group of biological tissues, and
d) comparing the at least one first calculated biological variable before bringing into contact with the at least one re-calculated second biological variable after bringing into contact, wherein a change in the at least one biological variable between first and repeated calculation indicates a biological effect of this active substance on the biological tissue.

29. A device for determining biological variables of a biological tissue or tissue equivalent in vitro, the device comprising:
a measuring unit for measuring an impedance spectrum Z(ω) of the biological tissue or tissue equivalent as a function of frequency to thereby obtain a measured impedance spectrum, and
an arithmetic unit programmed for the:
iterative matching of n model spectra Zm(ω)n obtained by modeling electrical variables to the measured impedance spectrum, wherein each Zₘ(ω)n is determined by a formula selected from the family of formulae:
Zₘ(ω)n=R_{S}+Z_{CPE}(ω)+Sum of (Z_{Cell}(ω)i) for i=1 to n,
where for each I
Z_{Cell}(ω)i is the impedance of an electrically active layer i in the tissue which corresponds to a parallel connection of an ohmic component R_{Cell}i of this layer and a real capacitive component C_{Cell}(ω)(Ni)i of this layer according to the formula:
Z_{Cell}(ω)i=R_{Cell}i∥C_{Cell}(ω)(Ni)i
and where
Ni is the ideality of the real capacitive component C_{Cell}(ω)(Ni)i and is always less than 1;
wherein, for each n, the model spectra Zm(ω)n are in each case approximated to the impedance spectrum Z(ω) measured in Step a) by variation of at least one of the impedance-determining components R_{Cell}i and capacitive component C_{Cell}(ω)(Ni)i of each electrically active layer i in each case;
determining matched model spectra for each model spectrum Zₘ(ω)n the model spectrum which for each n is optimally matched to the measured impedance spectrum Z(ω), wherein the optimally matched model in each case has a smallest residue compared with the measured impedance spectrum in each case; and
calculating at least one variable of the biological tissue or tissue equivalent by calculating at least one variable selected from ohmic component R_{Cell} and real capacitive component of each optimally matched model spectrum as the variable which characterizes the biological tissue or tissue equivalent.

30. The device according to claim 29, further comprising:
a bioreactor for cultivating biological tissue in vitro with electrodes for applying voltage/current and measuring the impedance over the cultivated biological tissue.

31. The device according to claim 30, wherein the bioreactor includes:
compartments for parallel separate cultivation of a plurality of biological tissues, and
electrodes associated with each compartment individually.

32. The device according to claim 30, wherein the bioreactor includes:
a multiplexer which connects a plurality of electrodes to the measuring unit for sequential measurement of the impedance spectra in the plurality of parallel cultivated biological tissues.

33. The device according to claim 29, further comprising a computer program including instructions for automatically carrying out:
a) measuring an electrical impedance of the biological tissue as a function of frequency, wherein a measured impedance spectrum Z(ω) is obtained;
b) matching of n model spectra Zₘ(ω)n to the measured impedance spectrum Z(ω), wherein each Zₘ(ω)n is determined by a formula selected from the family of formulae:
Zₘ(ω)n=R_{S}+Z_{CPE}(ω)+Sum of (Z_{Cell}(ω)i) for i=1 to n,
where for each i
Z_{Cell}(ω)i is the impedance of an electrically active layer i in the tissue which corresponds to a parallel connection of an ohmic component R_{Cell}i of this layer and a real capacitive component C_{Cell}(ω)(Ni)i of this layer according to the formula:
Z_{Cell}(ω)i=R_{Cell}i∥C_{Cell}(ω)(Ni)i
and where
Ni is the ideality of the real capacitive component C_{Cell}(ω)(Ni)i and is always less than 1;
wherein, for each n, the model spectra Zm(ω)n are in each case approximated to the impedance spectrum Z(ω) measured in Step a) by variation of at least one of the impedance-determining components R_{Cell}i and capacitive component C_{Cell}(ω)(Ni)i of each electrically active layer i in each case;
c) determining for each model spectrum Zₘ(ω)n the model spectrum which for each n is optimally matched to the measured impedance spectrum Z(ω), wherein the optimally matched model in each case has a smallest residue compared with the measured impedance spectrum in each case; and
d) calculating at least one variable selected from ohmic component R_{Cell} and real capacitive component of each optimally matched model spectrum as the variable which characterizes the biological tissue.
